(19) United States
(12) Patent Application Publication
(10) Pub. No.: US 2026/0216094 A1
(43) Pub. Date: Jul. 30, 2026

(54) BILAYER MEMBRANE THAT PROMOTES BONE REGENERATION AND PREVENTS POST-OPERATIVE COMPLICATIONS

(71) Applicants: UNIVERSIDAD DE LA FRONTERA, Temuco (CL); UNIVERSIDAD DE ANTOFAGASTA, Antofagasta (CL)

(72) Inventors: Víctor Javier BELTRÁN VARAS, Temuco (CL); Francisca ACEVEDO CANALA-ECHEVARRÍA, Temuco (CL); Iván Manuel VALDIVIA GANDUR, Antofagasta (CL); Claudia Andrea SANHUEZA SANHUEZA, Temuco (CL); Bernardo Arturo VENEGAS ROJAS, Viña del Mar (CL)

(21) Appl. No.: 19/144,288

(22) PCT Filed: Dec. 27, 2023
(86) PCT No.: PCT/CL2023/050141

§ 371 Date: Jun. 27, 2025

Related U.S. Application Data
(60) Provisional application No. 63/477,297, filed on Dec. 27, 2022.

(51) Int. Cl. A61K 9/70; A61K 9/14; A61K 31/593; A61K 33/38; A61K 47/34

(52) U.S. Cl. CPC A61K 9/7007; A61K 9/14; A61K 31/593; A61K 33/38; A61K 47/34

(57) ABSTRACT

A bilayer membrane that promotes bone regeneration and prevents postoperative complications, is resorbable, and has an antibacterial effect, composed of: an outer layer; and an inner layer that is located in contact with the outer layer and in contact with the area to be treated, where the outer layer of the membrane comprises a polyhydroxybutyrate (PHB)-based polymer and silver nanoparticles (AgNP), and the inner layer of the membrane comprises polycaprolactone (PCL) loaded with calcitriol.

### FIELD OF THE INVENTION

The present invention relates to the field of regenerative medicine. In particular, the present invention relates to treatment with dental implants, which promotes bone regeneration, for use in the regenerative treatment of peri-implantitis and bone defects in animals and humans, through a bilayer, resorbable membrane, with antibacterial and osteogenic effect.

Dental implant treatment allows the roots of missing teeth to be replaced, restoring the function and aesthetics of those teeth that have been lost for various reasons. Dental implants are devices, generally made of titanium and shaped like screws, which are surgically placed in the bone of the oral and maxillofacial region, osseointegrated, and can be rehabilitated with the use of prostheses. Where osseointegration is a direct, structural and functional connection between living, ordered bone and the surface of an implant subjected to functional loading.

Currently, osseointegrated implants have revolutionized dental rehabilitation and maxillary reconstruction. The success of this type of treatment in both edentulous and partially edentulous patients has been proven through solid experimental studies. Despite the risk factors after dental implant placement, the positive results achieved with this technique for oral rehabilitation of the patients and the high survival rates of osseointegrated implants have made this procedure one of the most recommended by dentists and specialists to replace the treatment of patients requiring removable partial dentures or fixed bridges. However, even under the best aseptic conditions, with optimal planning and preparation of the technique, some implants cannot be osseointegrated, given the wide variety of responses of people or animals to these treatments and their own health conditions.

After implant placement, a series of immediate, medium- and long-term complications may occur, which in most cases are not serious and are expected. These complications are due to the inflammatory process associated with the surgical procedure itself.

Some of the most common postoperative complications include: hemorrhage, edema, hematoma, pain, infection, neuralgia, anesthesia, paresthesia, suture dehiscence, implant loosening, implant fracture, implant rejection, allergic responses, among others.

As medium- or long-term complications of the placement of an osseointegrated implant, peri-implant diseases such as mucositis and peri-implantitis may occur.

Mucositis is defined as a reversible inflammatory process of the soft tissues surrounding a functioning implant, with clinical characteristics similar to gingivitis, with swelling, redness, bleeding and in some cases suppuration upon careful peri-implant probing, without loss of peri-implant supporting bone tissue.

Peri-implantitis is a pathological condition that occurs in the tissues surrounding osseointegrated implants, characterized by inflammation of the peri-implant mucosa and progressive loss of supporting bone. Upon clinical examination, bleeding and/or suppuration is present after careful peri-implant probing.

Over the years of analyzing the causes of pathological alterations in peri-implant support tissues, it has been concluded that this is multifactorial, but that bacterial infection is possibly the main etiopathological factor. The lack of connective fiber insertion and the reduction of vascular supply around the implant result in greater susceptibility to inflammation induced by bacterial plaque. Due to reduced vascularization and the parallel orientation of collagen fibers, peri-implant tissues are more susceptible to inflammatory disease than the soft tissues around the teeth.

The treatment of peri-implantitis, regardless of the method used, must consider the elimination of the infection and the restoration of the original condition of the peri-implant tissues to create the ideal conditions that allow promoting re-osseointegration in the affected implant, understanding re-osseointegration as the formation of new bone on an implant surface previously contaminated with a biofilm.

The treatment of peri-implant disease is one of the greatest challenges in oral implantology today. As with all diseases, the stage prior to pathology is key, as the goal is to prevent the disease from occurring, hence the fundamental importance of prevention and regular patient check-ups with a qualified professional.

### STATE OF THE ART

Currently, synthetic resorbable membranes have been widely used in medicine and dentistry for guided bone regeneration due to their high biocompatibility and ability to promote wound healing, avoiding surgery. However, once they are exposed or infected during the healing phase, they are extremely complex to manage.

There are several different synthetic resorbable membranes available (Lee & Kim et al., 2014; Zhang et al., 2022; Alauddin et al., 2022) for conventional guided bone regeneration procedures or around implants, with a barrier effect of 1 to 38 weeks according to the manufacturer, which are used in combination with a variety of bone grafts/substitutes (Sbricoli et al., 2020). However, the complication rate of membranes used in guided bone regeneration varies and depends on the type of procedure, the presence or absence of infection at the anatomical site, the stability and quality of the soft tissues covering the wound, oral hygiene, medical history, smoking habits, wound dehiscence, or the openness of the incision line, among other factors. For example, in relation to the type of procedure for vertical guided bone regeneration, complication rates during the healing period are reported ranging from 15 % to 21.1 % with the use of non-resorbable and resorbable membranes, respectively, considering that the sites to be treated did not present previous local infections (Cucchi et al., 2017).

Membrane exposure is generally considered the most common drawback of guided bone regeneration procedures and, therefore, the indication of systemic antibiotics, antibacterial mouthwashes or gel with local application for variable periods of time are frequently used by clinicians to avoid complete failure of regenerative therapy (Almutairi, 2018, García et al., 2018). However, in most cases where there is exposure to different agents by the membrane, it results in surgical removal of the membrane and failure of the therapy (García et al., 2018; Fontana et al., 2011). In this sense, it would seem that the development of a membrane with antibacterial properties at the time of accidental exposure, mainly during the early healing period (2-3 weeks), would favor optimal closure of the soft tissue over the membrane with a low risk of infection and, in addition, would prevent the proliferation of inflammatory tissues within the regenerated site.

The consequences of exposing the membrane to various agents range from minor problems such as membrane shedding and incomplete bone growth to major problems such as treatment failure, implant loss, and patient suffering. (Jovanovic, Spiekermann and Richter, 1992; Fontana, Maschera, Rocchietta and Simion, 2011).

In this same context, for the regenerative therapy of peri-implantitis, resorbable membranes have been preferably used (Sarmiento et al., 2018), and studies have focused mainly on incorporating antibiotics into bone grafts or developing antimicrobial properties around implants to prevent peri-implantitis (de Ávila et al., 2019). For example, recently Mercado, Hamlet and Ivanovski (2018), used a collagen membrane in combination with bone graft mixed with doxycycline powder, reporting a success rate of 56.6% of treated implants after 36 months.

Based on the scientific evidence presented above, it is clear that peri-implantitis constitutes a global health problem that no one is unaware of and that is far from being understood and managed appropriately (Pouponneau et al., 2020).

Among the known patents is U.S. Ser. No. 11/134,687, which discloses a method for preparing dental and orthopedic acrylic materials with antimicrobial properties using copper nanoparticle (CuNPs) technology diffused or arranged on said material, but this document does not disclose the bilayer with antimicrobial properties and osteogenic capacity. It is evident that the present invention has a superior technical effect over that declared in the state of the art.

Another document is patent U.S. Ser. No. 10/359,445, which discloses bioresorbable osteoconductive compositions and methods of using the composition as a scaffold for bone repair in periodontal, alveolar or maxillary regeneration, cranial bone defects and spinal regeneration. Bioresorbable compositions contain a bioresorbable polymer, a micro-or nanoparticle filler, and a pore-creating substance. The micro or nano filler can be any natural biocompatible material such as a metal, calcium carbonate, carbon, a biocompatible synthetic material, or a bioceramic such as hydroxyapatite. The pore-creating substance may be an effervescent agent such as a carbonate and an acid, but this document does not disclose a bilayer with antimicrobial properties and osteogenic capacity. It is evident that the present invention has a superior technical effect over that declared in the state of the art.

U.S. Ser. No. 15/317,306 discloses electrodynamically obtained fibers containing drugs and compositions comprising said fibers for application to the skin, lips or mucous membranes to deliver a specific amount of one or more pharmacological substances to the skin or mucous membranes. The formulation principle ensures the presence of the bioadhesive substance in the fibers in the form of undissolved material, allowing bioadhesion in situ by coming into contact, for example, with saliva in the oral cavity. The fibers are in the form of a layer and may be provided with one or more additional layers, for example, a backing layer that is insoluble in water or saliva and/or a layer that can influence the release of the drug from the final composition. Furthermore, the compositions are suitable for local application to moist internal surfaces such as the vocal cords or intestine, for example, for the treatment of inflammatory bowel disease. In particular, the invention relates to compositions comprising electrodynamically obtained fibers for application in the oral cavity to administer a pharmacological substance to the oral mucosa, but this document does not disclose a bilayer with antimicrobial properties and osteogenic capacity. It is evident that the present invention has a superior technical effect over that declared in the state of the art.

### SOLUTION TO THE TECHNICAL PROBLEM

Given the above, the development of new biomaterials with antibacterial potential and bone regeneration promoters is imperative for their potential use in the regenerative treatment of peri-implantitis and bone defects in animals and humans.

### BRIEF DESCRIPTION OF THE INVENTION

The present invention is a biocompatible and biodegradable, resorbable bilayer membrane with antibacterial and osteogenic capacity produced by the electrospinning technique (under the operational conditions of: voltage 25 kV, distance between the needle and the collector of 15 cm, flow 2 mL/h, thus obtaining micrometric-sized fibers with a thickness of 180 μm), which promotes bone regeneration, for its potential use in the regenerative treatment of peri-implantitis and bone defects in animals and humans.

This bilayer membrane is composed of an outer layer and an inner layer.

The outer layer of the membrane has antibacterial activity based on polyhydroxybutyrate (PHB) fibers in a range between 5-12% w/w and silver nanoparticles (AgNPs) at a concentration between 1 to 10% w/w, to prevent the formation of biofilms of Staphylococcus aureus, Streptococcus mutans, Pseudomona aeruginosa, Gandida albicans and Veillonella parvula, among others, during the healing period.

The inner layer has osteogenic capacity and is composed of polycaprolactone (PCL) fibers between 8 to 16% w/w loaded with calcitriol at a concentration between 0.1 to 2.0% w/w (dry weight).

### DESCRIPTION OF THE FIGURES

FIG. 1.A shows a Scanning Electron Microscopy (SEM) image of the bilayer without bioactives.

FIG. 1.B shows an EDX image of the bilayer loaded with bioactives (AgNPs and Calcitriol).

FIG. 1.C shows silver nanoparticles (AgNPs) in the bilayer by Transmission Electron Microscopy (TEM)

FIG. 2 shows a tensile test of the bilayer without bioactives and with bioactives.

FIG. 3 shows antibiograms obtained from a) S. aureus, b) S. mutans, c) P. aeruginosa and d) C. albicans on the bilayer membrane without bioactives and with bioactives (AgNPs and calcitriol).

FIG. 4 shows an evaluation of the antibiofilm activity of the bilayer on microorganisms after 24 hours of culture.

FIG. 5 shows the evaluation of planktonic growth of bacteria and yeast on the bilayer membrane with and without bioactives (AgNPs and calcitriol). The microorganisms were loaded at a concentration of 1×10⁷ CFU/mL in each sample.

FIG. 6.A. shows decontamination with titanium brushes.

FIG. 6.B. shows photodynamic therapy.

FIG. 7 shows microphotographs via Confocal Laser Scanning Microscopy (CLSM), Live/Dead staining.

FIG. 7.A shows a contaminated device.

FIG. 7.B shows a decontaminated and disinfected device.

FIG. 8 shows the micro-CT analysis of the application of the bilayer membrane on the peri-implant bone defect at 15, 30 and 60 days, where the great osteogenic capacity, incremental as a function of time, of the inner layer of the membrane composed of PCL and calcitriol can be observed.

FIG. 9 shows the micro-CT analysis of the application of the bilayer membrane at 15 and 30 days vs. the control group (peri-implant bone defect without regenerative treatment) where it can be observed that the superior technical effect in relation to the great osteogenic capacity of the bilayer membrane (red arrows) vs. the control group, generates a considerably greater volume of mineralized bone tissue and areas of re-osseointegration.

FIG. 10 shows the descriptive BS-SEM histology of the bilayer membrane application (15-30-60 days). The quality of the formed bone observed is equivalent to that of mature bone trabeculae, with wide medullary and vascular spaces, all generated under the bilayer membrane and at the site of the peri-implant bone defect.

FIG. 11 shows a time diagram of the evolution of a regenerative treatment using a bilayer membrane without bone graft, in a peri-implant bone defect (PIBD), with a titanium device contaminated with a biofilm simulating a peri-implantitis microcosm and which was treated by mechanical cleaning plus photodynamic therapy. CB: cortical bone, BM: bone marrow.

### DETAILED DESCRIPTION OF THE INVENTION

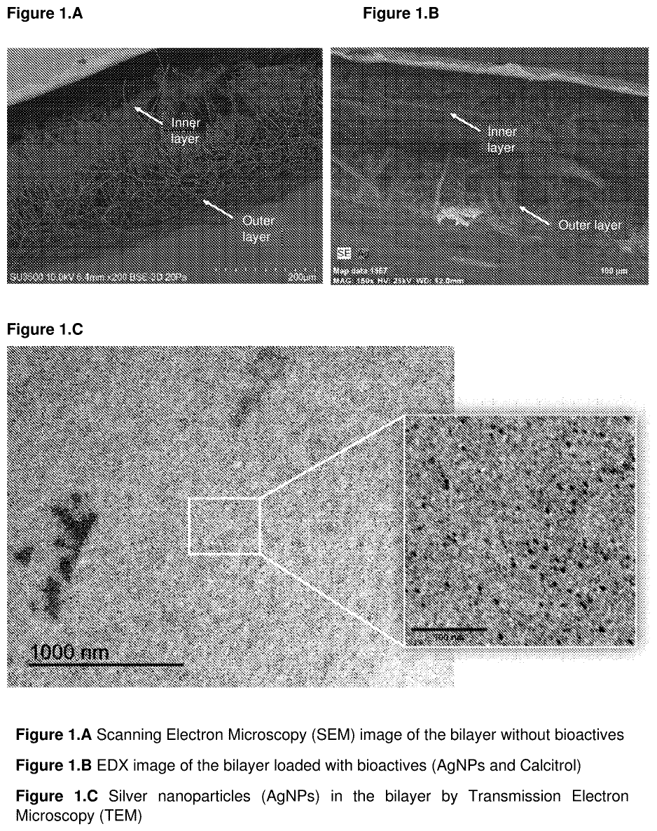

The bilayer membrane proposed here is designed for the prolonged release of therapeutic agents, including silver nanoparticles (AgNPs) and calcitriol, which, on the one hand, the action of AgNPs inhibits or reduces bacterial proliferation in the external area of the membrane and, on the other hand, calcitriol promotes bone regeneration, for example, around the dental implant or in a bone defect. This bilayer membrane modulates what occurs naturally in the healing process of oral soft tissue and peri-implant bone tissue. In this context, a bilayer membrane is developed using the electrospinning technique (FIG. 1).

To carry out the development of the inner membrane, polycaprolactone (PCL) is dissolved in chloroform between 8 to 16% w/w loaded with calcitriol at a concentration between 0.1 to 2.0% w/w dry weight.

This solution is electrospun under the following operating conditions of the electrospinning equipment: voltage 25 kV, distance between the needle and the collector of 15 cm, flow 2 mL/h, thus obtaining micrometric-sized fibers with a thickness of between 100 and 300 um.

For the development of the outer layer of the membrane, the polyhydroxybutyrate (PHB)-based polymer is first dissolved in chloroform in a range between 5-12% w/w and silver nitrate (AgNO₃) in N,N-dimethylformamide (DMF) for a final concentration of 1 to 10% w/w. Subsequently, the polyhydroxybutyrate (PHB) solution in chloroform and the silver nitrate (AgNO₃) solution in N, N-dimethylformamide (DMF) are mixed in a 9:1 ratio. This mixture is stirred for 7 days until it reaches a brown color, indicating the change in the oxidation state of silver and the formation of silver nanoparticles (AgNPs). This obtained solution is electrospun under the same operating conditions described above, thus obtaining micrometric-sized fibers with a thickness between 100 and 300 um that allow cell settlement. The chemical composition of the outer layer prevents the infiltration of epithelial cells and gingival connective tissue into the bone defect during the initial stage of regeneration, allowing the creation of an aseptic environment for bone regeneration, that is, a barrier effect that prevents the passage of non-bone cells into the bone defect beneath the membrane.
1.—A bilayer membrane, which promotes bone regeneration and prevents postoperative complications, resorbable, with antibacterial effect, CHARACTERIZED in that it comprises:
an outer layer; and
an inner layer that is located in contact with the outer layer where the outer layer of the membrane comprises a polymer based on polyhydroxybutyrate (PHB) and silver nanoparticles (AgNPs), and the inner layer of the membrane comprises polycaprolactone (PCL) loaded with calcitriol.
2.—The bilayer membrane of claim 1, CHARACTERIZED in that the concentration of the polyhydroxybutyrate (PHB)-based polymer of the outer layer is present in a range between 5-12% w/w.
3.—The bilayer membrane of claim 1, CHARACTERIZED in that the concentration of silver nanoparticles in the outer layer is 1 to 10% w/w (dry weight).
4.—The bilayer membrane of claim 1, CHARACTERIZED in that the polyhydroxybutyrate (PHB) in chloroform and silver nitrate (AgNO3) in N, N-dimethylformamide (DMF) are present in a 9:1 ratio for the formation of silver nanoparticles.
5.—The bilayer membrane of claim 1, CHARACTERIZED in that the concentration of polycaprolactone (PCL) in the inner layer is present between 8 to 16% w/w.
6.—The bilayer membrane of claim 1, CHARACTERIZED in that the calcitriol concentration of the inner layer is present between 0.1 to 2.0% w/w (dry weight).
7.—Use of the bilayer membrane of claim 1, CHARACTERIZED in that it serves to prepare a medical element useful in the treatment of bone defects in humans and animals.
8.—Use of the bilayer membrane of claim 1, CHARACTERIZED in that it serves to prepare a medical element useful in the regenerative treatment of bone lesions produced by peri-implantitis, osteomyelitis, osteonecrosis, post-extraction defects and implants placed in fresh alveoli, dehiscence-type defects and fenestrations around implants, bone augmentation of the alveolar ridge, bone defects following root resection, cystectomies and removal of impacted teeth, tumors, and for the regenerative treatment of the maxillary sinus, other paranasal cavities or bone defects in the walls of the skull base.
9.—Use of the bilayer membrane of claim 1, CHARACTERIZED in that it serves to prepare a medical element useful in guided tissue regeneration in periodontal defects.

### APPLICATION EXAMPLES

Below, examples of application of this patent application will be presented. These examples are provided for illustrative purposes only to provide a better understanding of the invention, but in no case should they be considered as limiting the scope of the protection sought. Additionally, specifications of different technical characteristics described in the examples may be combined with each other, or with other previously described technical characteristics, without this limiting the scope of the protection requested.

### Example 1: Method of Obtaining the Membrane

The bilayer membrane was produced using NEU-BM electrospinning equipment. The manufacturing process involved the manipulation of several controlled parameters, including the concentration of the polymers PCL (8% to 16% w/w) and PHB (5% to 12% w/w), as well as the concentration of the bioactives calcitriol (0.1% to 2.0% w/w) and AgNPs (1.0% to 10.0% w/w). In addition, the injection flow rate (0.1 mL/h to 2.5 mL/h) and voltage (10 kV to 25 kV) were adjusted in the electrospinning process.

It is important to note that the electrospinning process was carried out under normal ambient conditions, without the need to modify the pressure, temperature, or relative humidity of the environment. To achieve the required membrane thickness, the spinning process kept running for a period of 6 to 8 hours.

### Example 2: Membrane Characterization

Fiber size was monitored by scanning electron microscopy (SEM) using a HITACHI SU-2500 variable pressure equipment. 100 fiber diameter measurements were carried out per field. To evaluate the size of the AgNPs, transmission electron microscopy (TEM) was used with a Jeol JEM 1010 microscope at 100 kV. In addition, the bilayer membrane was characterized by X-ray diffraction in an Xpert Pro device. The analysis was performed at a voltage of 40 kV and 35 mA, using a monochromatic copper radiation lamp with a wavelength of 0.15406 nm. The scanning range was between 10 and 70° and at a rate of 2°/min.

### Example 3: Evaluation of the Mechanical Properties of the Bilayer Membrane

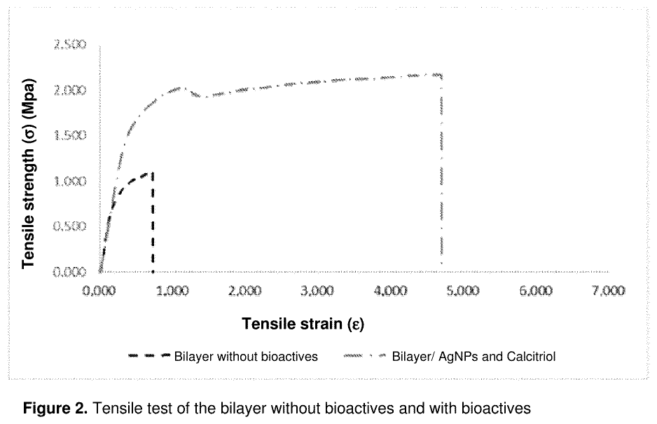

The bilayer membrane, when used in the bone defect in an animal model, has proven to be highly maneuverable without suffering any type of breakage when manipulated. This maneuverability is evidenced through the mechanical properties of the bilayer membrane (FIG. 2), by quantifying the response of the bilayer membrane when it is subjected to an external force, which triggers a tension generating a deformation in the material. The mechanical properties of the bilayer membrane were measured on a digital texturometer model CT3-10KG00000084 (Brookfield, UK). The membrane was cut into 4 cm² pieces which were placed in the equipment and subjected to the tension test at 0.1 mm/s and an initial gap of 8 mm. The parameters evaluated were Young's modulus, tensile strength and elongation at break.

The bilayer membrane has tensile strength values of 2.20±0.07 MPa, meaning that if this value is exceeded, the material will break. The elastic modulus was 5.08±0.66 MPa, which parameter indicates the rigidity of a material, that is, the more rigid a material is, the greater its elastic modulus. The elongation at break was 600.38±188.69%, which measures the increase in length of the bilayer membrane when it is subjected to a tensile stress before breaking. These values exceed those obtained for a commercial reference membrane, referring to a resorbable collagen membrane (BioMend, Zimmer, USA), for which a tensile strength of 11.294±1.198 MPa, an elastic modulus of 14.254±0.552 MPa and a lower elongation at break were obtained, reaching only 196±75.55%. These results demonstrate that the bilayer membrane has a higher elongation at break and is less rigid than the commercial reference membrane.

Tensile strength values ranging from 4.6 MPa to 10.4 MPa under dry conditions have been reported in the literature for commercial membranes used for guided bone regeneration in dental pathologies, such as Bio-Gide and Ossix Plus (Raz et al, 2019).

The mechanical properties of the bilayer prevent it from deforming or collapsing during the regenerative process, thereby maintaining space and protecting the surgical site, promoting the formation of new bone.

This bilayer also maintains an interface between the keratinized soft tissue and the bone defect. Regarding membrane maneuverability, exercises were performed on a phantom/model specially designed to simulate membrane manipulation over a peri-implant bone defect. In this context, 10 dental specialists participated, experts in performing regenerative implant procedures with an average of 19 years of experience as specialists, to whom a survey was applied with 7 questions with a Likert scale of 1 to 10 (1 being the lowest score and 10 the highest of satisfaction), related to the clinical maneuverability of the bilayer membrane. The results obtained from the questions were as follows: Is the manipulation of the membrane during placement in the bone defect adequate using routine surgical instruments? (8.8); Did the membrane not suffer deformation or tears in a controlled traction with 2 instruments (forceps)? (9.4); Is the stiffness of the SaffOss Plus membrane adequate to contain particulate bone graft in an open (flap) approach? (9.3); Can the SaffOss Plus membrane be sutured properly using a monofilament suture? (9.6); Once the SaffOss Plus membrane is wetted, is it easy to maneuver? (9.3); Once the SaffOss Plus membrane is moistened, is its texture softer? (9.5); Is the membrane easy to cut (with scissors)? (9.8).

### Example 4: Evaluation of the Antibacterial Activity of the Bilayer Membrane

The outer layer of the bilayer has antibacterial activity, through the incorporation of silver nanoparticles (AgNPs), against Staphylococcus aureus, Streptococcus mutans, Pseudomonas aeruginosa strains and its action against Gandida albicans was also evaluated, among others. Antibacterial activity was evaluated using different techniques.

First, it was evaluated by developing antibiograms, to assess whether the concentration of silver nanoparticles (AgNPs) that diffuse into the agar inhibits the growth of microorganisms. Antibiograms were performed according to the specifications of the Clinical and Laboratory Standards Institute document M2-A9. Each strain was adjusted to 0.5 McFarland in a 0.9% w/v NaCl solution before being inoculated onto Müller Hinton Agar (MHA) plates. Three pieces of 6 mm diameter membranes were sterilized under UV light for 30 minutes on each side and placed on the MHA plates. The procedure was repeated three times for each strain. The plates were incubated upside down at 37° C. for 24 hours. Bioactivity against each strain was evaluated using the agar well diffusion method by measuring the inhibition zone by image processing using ImageJ software.

Subsequently, the biofilm-forming capacity of microorganisms on the bilayer surface was evaluated. Biofilms correspond to the formation of more resistant bacterial communities and represent the usual form of growth of microorganisms in nature and on the dental surface. The antibiofilm effect was evaluated in C. albicans, S. aureus, P. aeruginosa and S. mutans.

The pre-inoculums were prepared according to the microorganism to be evaluated.
P. aeruginosa and S. aureus were grown on MacConkey agar and blood agar, respectively, and incubated at 37° C. under aerobic conditions. After 24 h, the plates were stored at 4° C. 3 mL of Luria Bertani broth (LB) and Trypticase soy broth (TS) were inoculated with a colony of P. aeruginosa and S. aureus, and incubated for 16-20 h at 37° C.
S. mutans ATCC 25174 derivative was grown on Columbia agar supplemented with 7% sheep blood at 37° C. under aerobic conditions. Then, a colony was taken and inoculated into a sterile Falcan tube containing 3 mL of BHB and incubated for 20 h at 37° C.
C. albicans was grown on Sabouraud agar and incubated at 37° C. for 24 h. The plates were then stored at 4° C. A pre-inoculum was performed by inoculating a colony into a sterile Falcon tube containing 3 mL of RPMI and cultured for 16-20 h.

Each inoculum was then prepared to achieve an optical density sufficient to induce biofilm formation. The conditions were as follows:
a) S. aureus was diluted in TS broth to achieve an optical density at 600 nm (OD₆₀₀) of 0.05;
b) P. aeruginosa was diluted in LB broth to achieve an OD₆₀₀ of 0.01;
c) Various concentrations of S. mutans were used to initiate biofilm formation in brain-heart broth (BHB) over a 24-hour period. Subsequently, for the purpose of this study, S. mutans was diluted in BHB until reaching an OD₆₀₀ of 0.01.
d) C. albicans was diluted in RPMI to achieve an OD₆₀₀ of 0.5.

The membranes were cut into 6 mm pieces using a sterile dermal die with a diameter of 6 mm. To ensure sterility, the membranes were disinfected by UV irradiation for 30 minutes on each side. After the disinfection process, each piece of membrane was placed in a designated well of a 96-well plate.

Then, 200 μL of each inoculum was placed into a well containing the bilayer membrane. S. aureus, P. aeruginosa and S. mutans were incubated for 20 h at 37° C. and under static conditions. C. albicans was initially incubated for 1.5 h with RPMI medium under shaking at 200 rpm and 37° C. to promote biofilm formation and adhesion. The medium was then changed to 200 μL of yeast extract peptone dextrose (YEPD) broth and incubated for 24 h under static conditions at 37° C.

To ensure accurate evaluation, a bacteria-free control sample was included for each triplicate sample. This control group allowed for comparison and differentiation between the effects caused by the presence of bacteria and any potential changes that occurred solely due to the membrane.

Finally, the growth of planktonic cells, which are biofilm cells that are released and grow in the culture medium, thus colonizing new areas, was evaluated. Planktonic growth of microorganisms was evaluated in 24-well plates. In each well, 0.5×4 cm² samples were placed around the edge of the well without interfering with the light passing through. Each microorganism was prepared at a concentration of 1×107 bacteria/mL in its respective culture broth as follows:
P. aeruginosa and S. aureus were grown on MacConkey agar and blood agar, respectively, and incubated at 37° C. under aerobic conditions. After 24 h, the plates were stored at 4° C. The pre-inoculum was prepared considering the following conditions: 3 mL of Luria Bertani broth (LB) and Trypticase soy broth (TS) were inoculated with a colony of P. aeruginosa and S. aureus, and incubated for 16-20 hours at 37° C.
S. mutans ATCC 25174 derivative was grown on Columbia agar supplemented with 7% sheep blood at 37° C. under aerobic conditions. Then, a colony was taken and inoculated into a sterile Falcan tube containing 3 mL of BHB and incubated for 20 h at 37° C.
C. albicans was grown on Sabouraud agar and incubated at 37° C. for 24 h. The plates were then stored at 4° C. A pre-inoculum was performed by inoculating a colony into a sterile Falcon tube containing 3 mL of RPMI and cultured for 16-20 h.

Each plate was incubated at 37° C. for 24 h in a SPECTROStar Nano microplate reader (BMG LABTECH, Germany). An optical density reading at 600 nm was taken every 30 min to assess the proliferation of microorganisms over time.

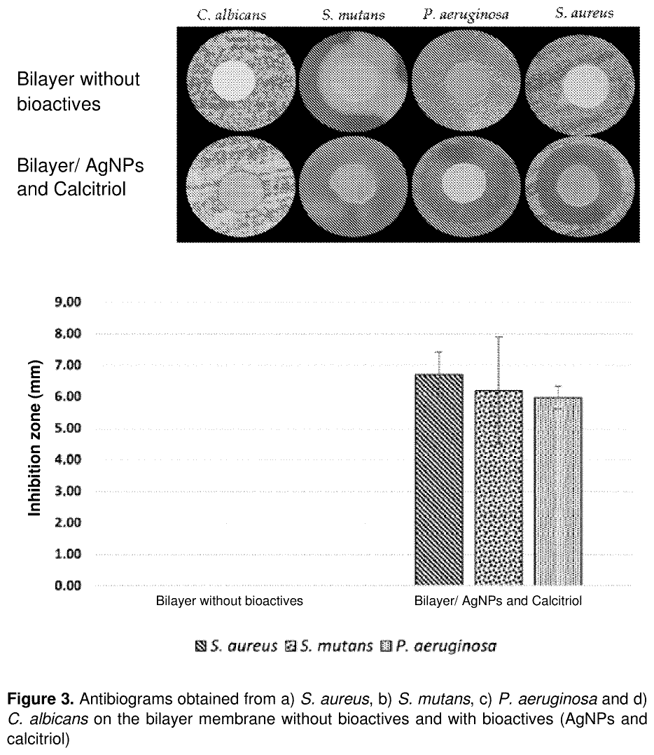

This analysis was developed to determine whether the concentration of silver nanoparticles (AgNPs) released from the bilayer membrane can inhibit the proliferation of planktonic microorganisms. Through the experiments carried out, it was observed that the concentration of silver nanoparticles (AgNPs) incorporated in the membrane is capable of diffusing about 12 mm in agar (FIG. 3), in addition to being able to prevent the proliferation of microorganisms in said area.

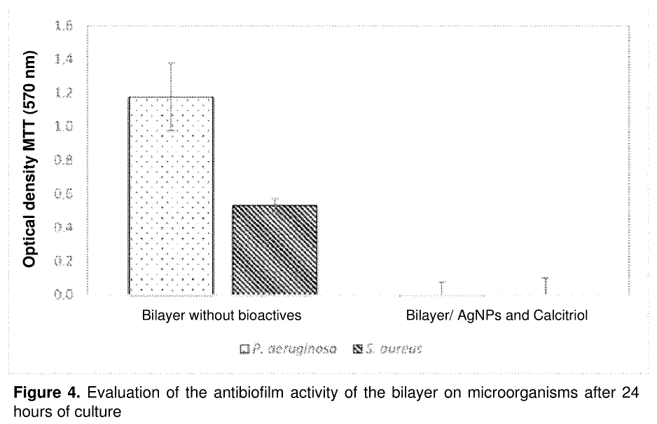

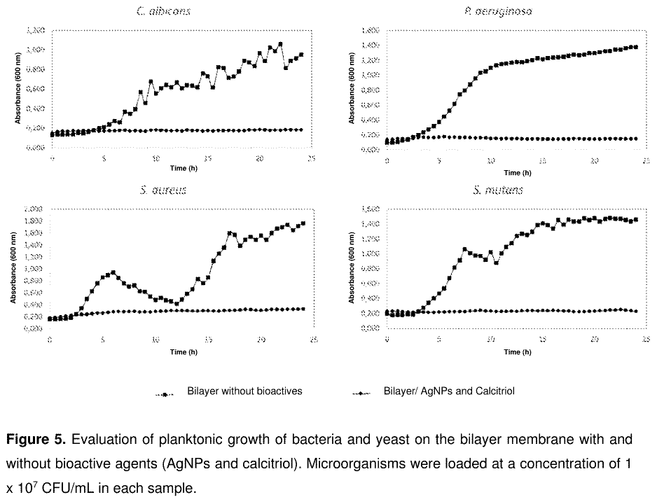

On the other hand, it is observed that the bilayer membrane prevents the formation of biofilm after 24 h of incubation (FIG. 4) and prevents the growth of planktonic bacteria (FIG. 5).

For its part, the bilayer membrane without bioactives allows planktonic growth of the four microorganisms evaluated, demonstrating the need to incorporate AgNPs at the concentrations studied to prevent the proliferation of microorganisms and the establishment of biofilms.

Based on the results obtained, it is possible to indicate that the antibacterial capacity of the bilayer membrane prevents the colonization of bacteria from the oral environment and the formation of bacterial biofilms on the membrane until optimal healing of the peri-implant bone and soft tissue is achieved, as well as inhibits the growth of planktonic bacteria, preventing bacterial recolonization.

Thus, the membrane's antibacterial effect is a viable alternative that reduces the use of local or systemic antibiotics in patients with peri-implantitis during the initial healing phase of peri-implant tissues in regenerative therapy.

### Example 5

Initially, a titanium device was designed and manufactured with the same surface treatment as the dental implant, which covers the upper portion of the implant and is secured by a central screw. This device is contaminated by a peri-implantitis biofilm generated under in vitro conditions. For this purpose, a pool of saliva from healthy patients was generated to form the microbial inoculum. Under anaerobic conditions, the titanium device immersed in a culture medium was incubated in vitro for 15 days. A characterization of the bacterial communities of the biofilm and the inoculum was carried out. 261 bacterial taxa (bacterial species, genera or related classifications) were identified in the saliva used for the biofilm inoculum, of which 32 have a relative abundance of at least 1% bacterial composition. Porphyromonadaceae and Prevotellaceae are the most abundant, also with a significant representation of bacteria of the genus Neisseria. Porphyromonas are periodontopathogenic bacteria, bacteria of the genus Prevotella are normally present in the oral cavity, as well as several commensal species of the genus Neisseria.The bacterial composition of the biofilm is lower in diversity than that of saliva. However, 72 taxa were identified, with a clear prevalence of Pseudopropionibacterium propionicum, which constitutes more than 43% of the biofilm microbiota; this bacteria has an abundance of 0.14% in saliva. Genera Veillonella and Eikenella are also dominant in the biofilm, although at a lower but significant abundance compared to P. propionicum. P. propionicum It is a species of bacteria commonly found in the human mouth and is of interest for its pathogenic capacity. It grows in the form of filaments and branches and could correspond to the filament structures observed by confocal microscopy. Eikenella corrodens (8.24% relative abundance) is a common peri-implant pathogen so its prevalence in the biofilm structure is expected. Likewise, Veillonella parvula (5.13% relative abundance) and Veillonella dispar (4.34% relative abundance) are bacteria identified as common components of the biofilm that forms dental plaque, where it has been described that they can co-aggregate with other microorganisms.

An experimental animal model was used in the tibia of a New Zealand rabbit. The sample size was estimated through simulation in STATA15 software considering the following assumptions: 6 treatment groups, the maximum value of capacity to form new bone at 30 days was 38 mm³, minimum value was 13 mm³, with a homogeneous standard deviation in the 6 groups of 4.38mm³ , according to the results of Trento et al. (2020) and with a uniform delta between groups of 5 mm³. The database generated by simulation was selected to be the size that allowed obtaining significant differences through the Kruskal Wallis test for multiple comparisons between groups separated by at least 2 groups. A post-hoc analysis was applied to this simulated base, obtaining a power of 0.99. The estimated sample size per group was 10 tibias, which allows a minimum size for applying both parametric and non-parametric tests.

The total number of animals was randomly divided into 2 groups (Group A: 15 animals, 30 tibias; Group B: 15 animals, 30 tibias). The 30 tibias in each group were randomized to receive 6 types of treatment for the peri-implant bone defect: group 1 received only the bilayer membrane; group 2 received a synthetic resorbable PLGA membrane (GUIDOR® ES Membrane Degradable Solutions AG, Switzerland); group 3 received the bilayer membrane plus synthetic bone graft (GUIDOR® Calc-i-oss Crystal, Collagen Matrix); group 4 received a synthetic resorbable PLGA membrane plus synthetic bone graft (SBG); group 5 received SBG without the membrane; and group 6 (control) received no regenerative therapy (spontaneous healing). Additionally, a positive control group was incorporated that consisted of the placement of contaminated titanium caps and did not receive regenerative therapy, and finally an experimental group was evaluated with the bilayer membrane without bone graft at 60 days. Euthanasia of animals in group A was performed after 15 days and group B after 30 days.

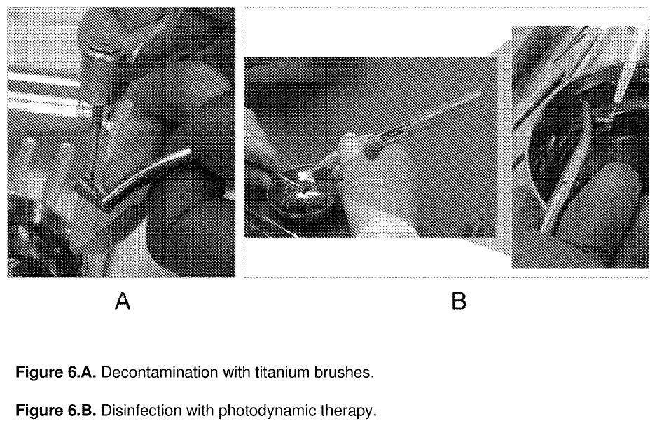

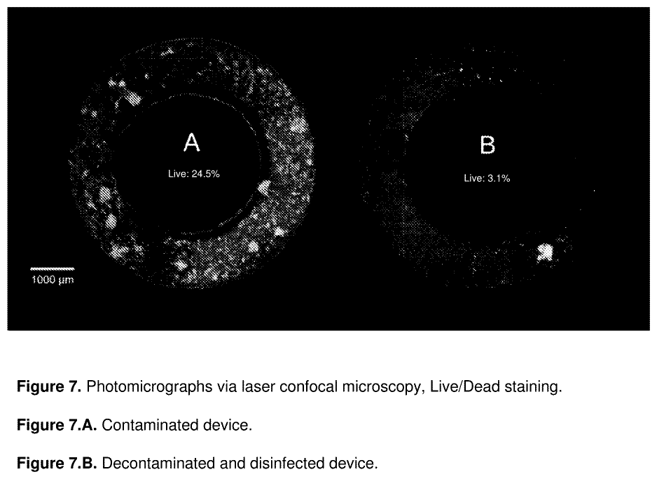

Once the detachment of a full-thickness flap (mucoperiosteal) has been performed, in parallel, the protocol for decontamination and disinfection of the contaminated surface of the titanium device by a biofilm of peri-implantitis microcosm is carried out, using titanium brushes (FIG. 6A) and subsequently photodynamic therapy is applied on the contaminated titanium surface, as indicated in FIG. 6B. This process was validated using laser confocal microscopy; the images obtained demonstrate the effectiveness of the decontamination and disinfection procedure, with a 3.1% of the surface of the remaining live biofilm.

The bone drilling protocol is then performed according to the manufacturer, for the installation of a standard implant of 3.7 mm diameter and 10 mm length (JDEvolution Implant, JDentalCare, Italy).

Once the implant is installed, the 7 mm diameter cortical bone window (critical bone defect) is removed to form the peri-implant bone defect using a trephine with continuous saline irrigation. At this point, the upper portion of the dental implant is covered with the titanium device containing a previously decontaminated/disinfected peri-implantitis biofilm, leaving the bone defect around a titanium surface with a reduced microbial environment simulation, allowing for standard regenerative therapy (synthetic resorbable membrane and bone graft) and promoting re-osseointegration of the peri-implant bone defect.

The present invention (bilayer membrane) is then applied to the peri-implant bone defect, with or without the use of a bone graft, and the bilayer membrane is secured by sutures to prevent its displacement over the bone defect.

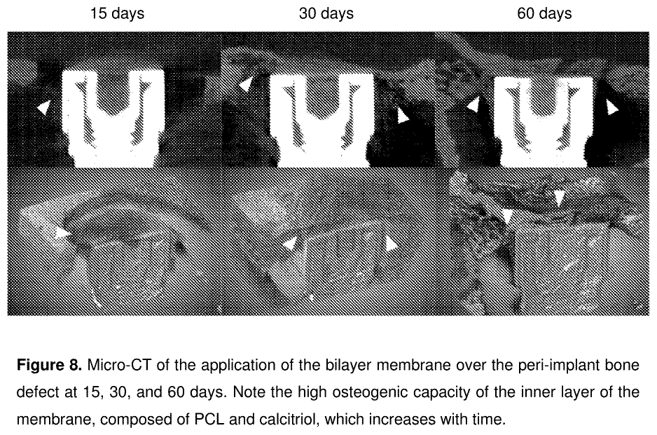

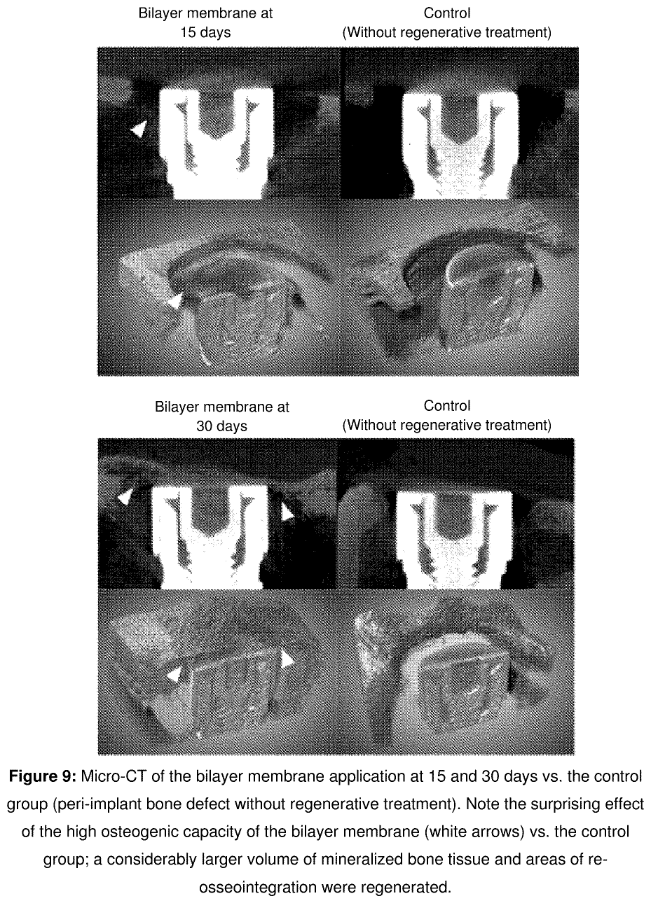

Finally, the flap is closed, leaving the implant under the membrane, which covers the entire peri-implant bone defect for a period of 15 to 30 days to promote bone regeneration around the implant. Additionally, an experimental group with the bilayer membrane without bone graft was evaluated at 60 days, as shown in FIG. 8. The micro CT images clearly show the formation of bone bridges towards the dental implant at 15 days (see arrow) and at 30 days, a partial filling of the bone defect can be identified and even a slight bone apposition on the cervical portion of the implant, demonstrating the high osteogenic capacity of the bilayer membrane without the need to use a bone graft, compared to the experimental group of the synthetic resorbable PLGA membrane without bone graft. At 60 days, a complete bone filling of the peri-implant defect can be clearly seen, and the presence of mature bone trabeculate can even be observed above the cervical portion of the implant. This finding demonstrates that considerable bone regeneration has been achieved under the bilayer membrane up to this evaluation time.

### Example 6: Ability to Form New Bone

To comparatively evaluate the ability of the bilayer membrane to form new bone in all treated groups, quantification of the newly formed bone was performed using a micro-CT scanner (SkyScan 1278) with a resolution of 51 μm. The values of mineralized tissue in the peri-implant bone defect (PIBD) were analyzed using 5 parameters: bone volume (BV), bone volume percentage (BV/TV), bone surface area (BS), bone surface area/volume ratio (BS/BV), and bone surface density (BS/TV).

In relation to the capacity to form new bone, the bilayer membrane significantly exceeds (P=0.042) the synthetic resorbable PLGA membrane, bone volume generated at 15 days (mean 15.47±2.46 versus 5.54±0.70) and at 30 days (mean 21.57±2.8 versus 7.23±2.36) with a P value=0.00. Similarly, the results for the bone surface/total volume ratio for the bilayer membrane at 15 days significantly exceed (P=0.029) the synthetic PLGA resorbable membrane (mean 2.00±0.36 versus 1.47±0.047) and at 30 days (mean 2.64±0.21 versus 1.66±0.21; P value=0.01). In relation to the capacity to form new bone, the bilayer membrane plus bone graft significantly outperforms the synthetic PLGA resorbable membrane plus bone graft (P=0.02), with a bone volume generated at 15 days (mean 43.3±7.87 versus 30.34±6.49) and at 30 days a total volume (mean 59.75±0.04 versus 59.67±0.01) with a P=0.07 value.

### Example 7: Bone Quality

An ultrastructural analysis of the newly formed peri-implant bone tissue was performed using backscatter electron microscopy (BS-SEM) at the different times of euthanasia of the animals. Immediately postmortem, tibial sections were obtained from the animals and covered with the bilayer membrane. The samples were then fixed in 10% neutral-buffered formaldehyde for 48 h. The bone segments were then prepared for embedding in light-curing resin (Technovit®). Subsequently, the embedded samples are cut along the central longitudinal axis of the implant using an Exact® diamond saw (0.2 mm thick) and processed for BS-SEM analysis (Manzanares et al. 1997; Franch et al. 2000; Beltran et al. 2020).

During BS-SEM analysis, a series of photomicrographs (50× to 150×) were obtained. The following parameters are mainly analyzed: Lamellar bone: This type of bone tissue exhibits osseous organization and is measured according to its lamellar apposition; Fibroreticular bone or primary bone tissue: This type of bone tissue has a regular bone structure with isolated and polygonal cellular spaces; Calcified chondroid tissue: This type of bone tissue has a characteristic appearance of calcified tissue with large, irregular, and confluent cellular spaces; Vascular or medullary spaces: Black spaces near calcified tissue compatible with spaces occupied by blood vessels or bone marrow (Beltrán et al. 2020).

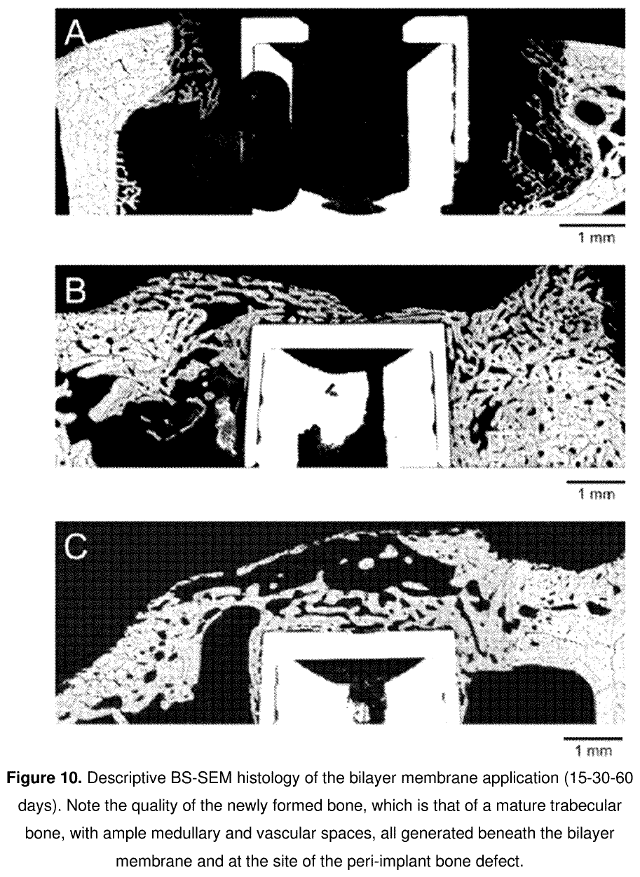

This series of images obtained from backscatter electron microscopy (FIG. 10) show the regeneration of peri-implant bone tissue favored by the bilayer membrane at 15, 30, and 60 days. At 15 days, the development of chondroid-type bone tissue is observed, which partially covers the bone defect, and at 30 and 60 days, a progressively greater amount of bone tissue is observed, mainly of the lamellar type (bone tissue has even formed above the head of the implant, immediately under the bilayer membrane), with the presence of vascular/medullary spaces, compatible with high-quality regenerated bone tissue with a good vascular supply. The formation of mature trabecular bone tissue can even be visualized immediately beneath the bilayer membrane at 60 days.

Regarding the quality and distribution of bone types within the peri-implant bone defect, this was evaluated through the total percentages of newly formed bone types identified through BS-SEM. Regarding the quality of the newly formed bone, the bilayer membrane shows the expected evolutionary trend in guided bone regeneration, from the predominance of chondroid bone as the initial newly formed bone, towards the primacy of lamellar bone as the mature newly formed bone, passing from one condition to the other through a temporally variable transition of fibroreticular bone. The expected proportion and transition from the literature, for the proposed animal model at 15 days are: chondroid bone: 60%, fibroreticular: 30% and lamellar bone: 10%. Therefore, the expected percentage weight ratio at 15 days was 6/3/1, respectively.

The bilayer membrane with graft, at 15 days shows the following average values for the total bone percentages: 1.59% for chondroid bone (range 0.46 to 2.93), 9.22% for fibroreticular bone (range 7.18 to 11.31), and 0.50% for lamellar bone (range 0.12 to 1.74); on the other hand, the synthetic resorbable PLGA membrane with bone graft presented the following values for the same variables: 0.24% chondroid bone (range 0.11 to 0.34), 1.47% fibroreticular bone (range 0.65 to 2.13) and 0.00% for lamellar bone (range 0.00). All these differences at 15 days are statistically significant between both membranes and are established in favor of the bilayer membrane with graft with a p value of 0.009 to 0.019, for the three types of bone in the Wilcoxon W and Mann-Whitney U tests. At 30 days, the values delivered by the bilayer membrane plus bone graft with an average of 0.32% chondroid bone (range 0.10 to 0.45), for the total percentage of bone present in the area at 30 days, 9.78% for fibroreticular bone (range 6.06 to 12.95) and 16.92% lamellar bone (range 12.35 to 21.47), thereby exceeding the values for the expected distribution of newly formed tissues. The synthetic resorbable membrane of PLGA plus graft, meanwhile, at 30 days presents values of 0.16% for the chondroid bone (range 0.00 to 0.28), 9.22% for the fibroreticular bone (range 4.82 to 15.37) and 16.30% for the lamellar bone (range 4.46 to 26.90), offering at first glance a similar behavior to the bilayer membrane at the percentage level, but with wider ranges and greater dispersion of data, which means that within the comparison with the set of groups with graft and controls, the bilayer membrane exceeds with a p value of 0.022 in the Kruskal-Wallis H test, the synthetic resorbable membrane of PLGA with graft, the graft alone, the group without treatment and the group with the contaminated cap (mean ranges of 17.00, 15.20, 6.25, 9.75 and 5.75, respectively), in the percentage of total lamellar bone generated at 30 days, and the p-value is a trend even for the percentage of fibroreticular bone at 30 days, which is 0.56.

Regarding the values mentioned above, in relation to the quality of the formed bone, the bilayer membrane shows an evolutionary trend, offering significant differences and values for chondroid and fibroreticular bone over the synthetic resorbable PLGA membrane at 15 days and lamellar bone at 30 days. For the bilayer membrane with graft versus synthetic resorbable PLGA membrane with graft, the same trend is observed, significant at 15 days, with the difference decreasing at 30 days. However, the evolutionary trend toward higher-quality bone formation in the bilayer membrane is clear and sustained, without resorption, which allows us to project a long-term osteogenic effect of the invention.

The evaluation of the BIC (Bone Implant to Contact) zone in the images of the samples processed through BS-SEM was carried out through a study unit (BAFO: Bone Area Fraction Occupancy) between two threads of the dental implant surface, in contact with the cortical bone area within the peri-implant bone defect. The % BIC of the bilayer membrane without bone graft was calculated at 30 days: 57.6±16.8 and the bilayer+bone graft membrane at 30 days: 55.5±11.1. In parallel, the percentage of bone filling (% Bf: bone filling percentage) was calculated in BAFO intercalated by vascular and medullary spaces, obtaining values for the bilayer membrane without bone graft at 30 days: 74.8±7.4 and the bilayer+bone graft membrane at 30 days: 65.4±15.8. This demonstrates the osteogenic capacity of the bilayer membrane itself without the need to use a bone graft in a critical peri-implant bone defect to promote re-osseointegration.

The histological results reveal a significant osteogenic effect of the bilayer membrane on the peri-implant bone defect, which progressively increases at different evaluation times.

### Example 8: Toxicity

In order to evaluate possible systemic toxicological effects of the bilayer membrane in experimental animals, serum biochemical and histological parameters were evaluated. For the evaluation of biochemical parameters, the blood sample was transferred to a tube without anticoagulant, carefully to avoid hemolysis. The blood was centrifuged at 1500 rpm for 10 min and the serum obtained was subsequently stored at −20° C. until analysis. Commercially available kits (Valtek Diagnostic) were used.

Both, the bilayer membrane group without bone graft (Albumin: 33.75 g/L; Calcium 9.88 mg/dL; Creatinine: 1.38 mg/dL; Urea: 31.5 mg/dL. ALT 3.55 U/L; LDH 28 U/L) as the bilayer membrane plus bone graft group (Albumin 32.2 g/L; Calcium 10.67 mg/dL; Creatinine: 2.35 mg/dL; Urea: 45.22 mg/dL; ALT: 4.7 U/L; LDH; 24.5 U/L), present values in biochemical parameters within the normal range. This allows us to rule out evidence of liver and kidney toxicity associated with the treatments given to the experimental animals.

For histopathological analysis, parameters considered included the presence of inflammation, as well as the possible presence of necrosis and fibrosis in liver tissue. The tissues were fixed in 4% buffered formaldehyde, processed and embedded in paraffin, then sectioned at a thickness of 5 μm and stained with Hematoxylin-Eosin, Masson's Trichrome and Giemsa.

From a qualitative point of view, the presence of lymphocytes in a non-pathological context was observed, with an absence of necrosis and fibrosis. In immunohistochemical analysis, the presence of blood vessels in the tissues detected by CD31 expression was associated with their normal vasculature, with no angiogenesis associated with inflammation observed. Similarly, positive Ki67 labeling was occasionally observed, ruling out pathological cell proliferation. The above observations allow us to rule out histological and immunohistochemical evidence of renal toxicity associated with the treatments administered to the experimental animals.

### Advantages of the Present Invention

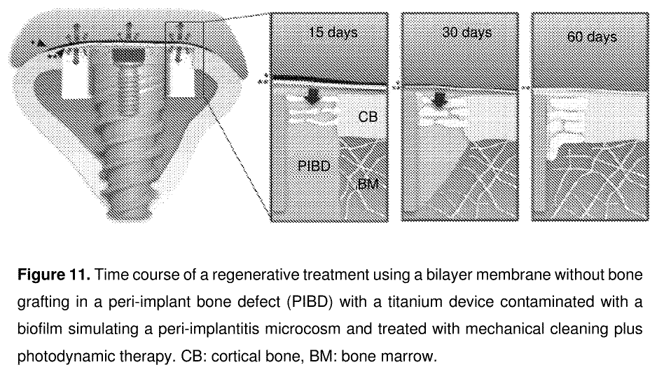

During the first few days, the outer layer of the bilayer membrane with antibacterial activity (FIG. 11, symbol layer*), progressively degrades, preventing on the one hand the formation of a biofilm on the membrane and also preventing, on the other hand, the proliferation of inflammatory tissues inside the peri-implant bone defect.

Thus, Favorable Scar Tissue Formation Over the Membrane Is Expected Within 3-4 Weeks.

On the other hand, the inner layer of the membrane with osteogenic activity (FIG. 11, symbol layer**), allows the bone tissue of the peri-implant defect to regenerate through the prolonged release of calcitriol from the membrane into the defect, promoting bone regeneration around the implant.

In this way, bone regeneration around the implant is significantly favored, over a period of at least 30 days in the experimental model.

In turn, the present invention establishes that it is not necessary to remove the membrane with a second surgical intervention, since the bilayer membrane gradually degrades over time, without generating underlying inflammatory processes.

To assess the degradation time of the bilayer membrane, vascularization in the area, the presence of collagen, multinucleated giant cells, and the immune response to Ki-67 were considered. For the histological identification of the membrane, an adequate form of distinction between the inorganic material and the adjacent connective tissue was sought, also considering the proximity to the bone surface, by staining the samples with hematoxylin-eosin, Masson's trichrome, and Ki-67 staining was also performed to evaluate proliferative activity. For this analysis, photographic records of 5 microscopic fields of each of the samples were obtained. The images were analyzed using ImageJ software, counting inflammatory cells, multinucleated giant cells, blood vessels, and Ki-67-positive cells.

The first parameter considered in this analysis is the percentage of vascularization present in the microscopic field of observation, in which case a progressive decrease in them can be expected as guided bone regeneration progresses and therefore connective tissue decreases. In this case, at 15 days, the average Microvascular Density (MVD) count for the bilayer membrane was 9.39%, while the synthetic PLGA resorbable membrane showed 11.11% at the same 15 days. At 30 days, a decrease can be observed in both cases, reaching up to 5.62% for the bilayer membrane and 9.11% for the synthetic PLGA resorbable membrane. This decrease reflects the improved conditions within the peri-implant bone defect in terms of bone tissue regeneration and maturity in favor of the bilayer membrane.

Regarding the presence of collagen tissue in the microscopic field studied in relation to the membrane, after 15 days, 0.280 mm² of collagen was observed for the bilayer membrane versus 0.301 mm² for the synthetic PLGA resorbable membrane. A reduction in these values over time is expected, considering the gradual process of new bone formation and the reduction of connective tissue around the defect being repaired, considering the tissue remodeling process that involves greater progress associated with a smaller amount of connective tissue. At 30 days, the observed reduction corresponds to a remaining 0.095 mm² of collagen in the case of the bilayer membrane versus 0.180 mm² of collagen for the synthetic PLGA resorbable membrane. It can be observed in this sense that the bilayer membrane behaves better than the synthetic resorbable PLGA membrane in terms of reducing the presence of collagen tissue in the microscopic fields at both observation times, which generates a more favorable condition for the bone regenerative process. Regarding the presence of multinucleated giant cells, which is a good indicator of active membrane degradation, the situation for the bilayer membrane at 15 days reports 5.8 detections per observed microscopic field, while for the synthetic resorbable PLGA membrane only 0.4 are detected. This same situation at 30 days shows a count of 13.8 for the bilayer membrane versus 5.8 for the synthetic PLGA resorbable membrane, which indicates an active and more permanent degradation process of the bilayer membrane compared to the synthetic PLGA resorbable membrane at both observation times.

The last evaluation considered was cell proliferation by immunohistochemistry, using the Ki-67 antibody, which expresses higher values whenever cell proliferation or infiltration has occurred and a progressive reduction in its values would be expected between 15 and 30 days. At 15 days, an average count of 18.4 was obtained for the bilayer membrane and 73 for the synthetic PLGA resorbable membrane, and at 30 days, an average count of 13 was obtained for the bilayer membrane versus 11.4 for the synthetic PLGA resorbable membrane. The above assumes that the bilayer membrane generates a first reaction of controlled cell proliferation at 15 days, which coincides with the degradation of the first layer of the membrane (external), while it is reduced significantly increased cell proliferation at 30 days, indicating favorable conditions for new bone formation.

These results suggest that the bilayer membrane undergoes a process of structural degradation between 15 and 30 days in the animal model. In this context, the bilayer membrane allows for the creation of an interface between the connective tissue and the developing bone tissue, thus promoting gradual osteogenic activity under the membrane, without interfering with tissue organization and/or architecture. These histological results confirm its gradual degradation and barrier effect to prevent the infiltration of inflammatory (non-bone) tissues into the peri-implant defect.

Considering that preventing early infections in bone regenerative therapies is critical to avoiding therapy failure, the characteristics of this new membrane make it a unique and unparalleled biomaterial in the regenerative medicine market, especially in its potential use for the regenerative treatment of peri-implantitis and bone defects in animals and humans.

Furthermore, due to the characteristics of this bilayer membrane, it can be applied to various bone regenerative medicine procedures, such as: reconstruction of bone defects in oral and maxillofacial surgery, peri-implant bone regeneration, bone ridge augmentation, periodontal and alveolar regeneration post-extraction, bone regeneration in traumatology, among others.

There are no membranes on the market today that meet the antibacterial and bone regeneration-promoting properties for use in regenerative treatments for peri-implantitis or similar. Furthermore, there is no evidence of membranes with bone regenerative capacity on their own, which do not require use in conjunction with bone grafts for guided bone regeneration.

1. A bilayer membrane, which promotes bone regeneration and prevents postoperative complications, resorbable, with antibacterial effect comprising:
an outer layer; and
an inner layer that is located in contact with the outer layer and in contact with an area to be treated, where the outer layer of the membrane comprises a polymer based on polyhydroxybutyrate (PHB) and silver nanoparticles (AgNPs), and the inner layer of the membrane comprises polycaprolactone (PCL) loaded with calcitriol.

2. The bilayer membrane of claim 1, wherein the concentration of the polyhydroxybutyrate (PHB)-based polymer of the outer layer is present in a range of 5-12% w/w.

3. The bilayer membrane of claim 1, wherein the concentration of silver nanoparticles in the outer layer is 1 to 10% w/w (dry weight).

4. The bilayer membrane of claim 1, wherein a ratio of polyhydroxybutyrate (PHB) in chloroform and silver nitrate (AgNO₃) in N,N-dimethylformamide (DMF), is 9:1 ratio for the formation of silver nanoparticles.

5. The bilayer membrane of claim 1, wherein the concentration of polycaprolactone (PCL) in the inner layer is 8 to 16% w/w.

6. The bilayer membrane of claim 1, wherein the calcitriol concentration of the inner layer is 0.1 to 2.0% w/w (dry weight).

7. A method of treating defects in humans and animals comprising applying the bilayer member of claim 1 to the humans and animals in need thereof.

8. Method for regenerative treatment of bone lesions produced by peri-implantitis, osteomyelitis, osteonecrosis, post-extraction defects and implants placed in fresh alveoli, dehiscence-type defects and fenestrations around implants, bone augmentation of the alveolar ridge, bone defects following root resection, cystectomies and removal of impacted teeth; tumors and for the regenerative treatment of the maxillary sinus, other paranasal cavities or bone defects in the walls of the skull base comprising preparing a medical element comprising the bilayer membrane of claim 1 and administering to a patient in need thereof for the regenerative treatment of bone lesions produced by peri-implantitis, osteomyelitis, osteonecrosis, post-extraction defects and implants placed in fresh alveoli, dehiscence-type defects and fenestrations around implants, bone augmentation of the alveolar ridge, bone defects following root resection, cystectomies and removal of impacted teeth; tumors and for the regenerative treatment of the maxillary sinus, other paranasal cavities or bone defects in the walls of the skull base.

9. A medical element useful in guided tissue regeneration in periodontal defects comprising the bilayer membrane of claim 1.
